(19) United States
(12) Patent Application Publication
(10) Pub. No.: US 2026/0226000 A1
(43) Pub. Date: Aug. 6, 2026

(54) Methadone Active Agents and Methods of Use Thereof

(71) Applicant: Ensysce Biosciences Inc., La Jolla, CA (US)

(72) Inventor: Lynn Kirkpatrick, La Jolla, CA (US)

(21) Appl. No.: 19/157,256

(22) PCT Filed: Mar. 26, 2024
(86) PCT No.: PCT/US2024/021447

§ 371 Date: Aug. 15, 2025

Related U.S. Application Data
(60) Provisional application No. 63/454,822, filed on Mar. 27, 2023.

(51) Int. Cl. C07C 271/20; A61K 31/27; A61K 31/40; A61K 31/4458; A61K 31/5375; C07D 207/09; C07D 211/26; C07D 265/30

(52) U.S. Cl. CPC C07C 271/20; A61K 31/27; A61K 31/40; A61K 31/4458; A61K 31/5375; C07D 207/09; C07D 211/26; C07D 265/30

(57) ABSTRACT

The present disclosure provides methadone prodrugs, pharmaceutical compositions thereof, and such prodrugs methods of use, where the pharmaceutical compositions comprise a methadone prodrug that provides enzymatically-controlled release of methadone, utilized for the treatment or prevention of pain, as well as methods for administering to a subject such prodrugs.

### CROSS-REFERENCE TO RELATED APPLICATION

This application is related to U.S. Provisional Patent Application Ser. No. 63/454,822 filed Mar. 27, 2023; the disclosure of which application is herein incorporated by reference.

### INTRODUCTION

Methadone is a synthetic opioid agonist that is often used for opioid maintenance therapy in opioid dependence and for chronic pain management. Opioids like methadone are susceptible to misuse, abuse, or overdose. Use of and access to methadone often needs to be controlled. The control of access to methadone is expensive to administer and can result in denial of treatment for patients that are not able to present themselves for dosing. For example, patients suffering from acute pain may be denied treatment with a pain drug unless they have been admitted to a hospital. Furthermore, control of use is often ineffective, leading to substantial morbidity and deleterious social consequences.

### SUMMARY

The present disclosure provides methadone active agents, pharmaceutical compositions, and their methods of use, where the pharmaceutical compositions comprise a methadone active agent and a pharmaceutically acceptable carrier.

In some embodiments include a methadone active agent that is a compound of formula ME-(I):

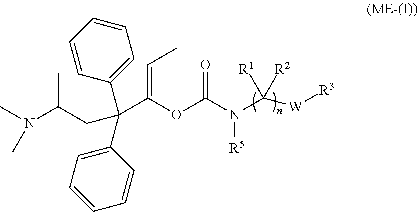

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(II):

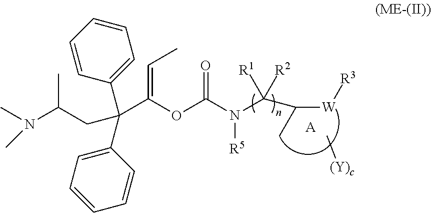

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIa):

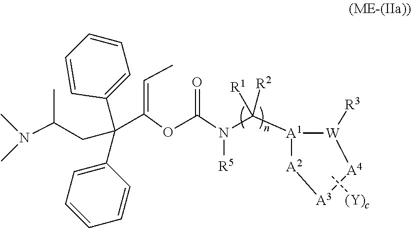

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, and A⁴ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIb):

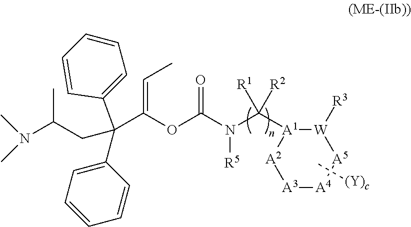

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, A⁴ and A⁵ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(III):

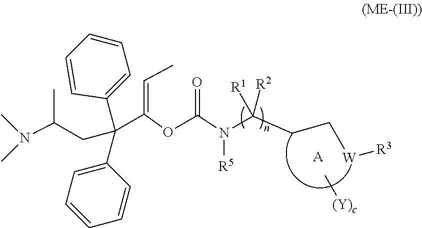

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIIa):

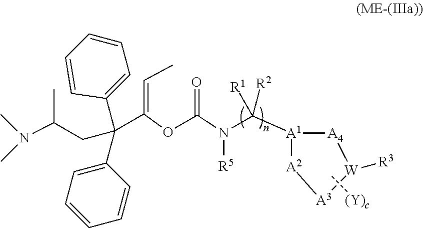

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, and A⁴ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIIb):

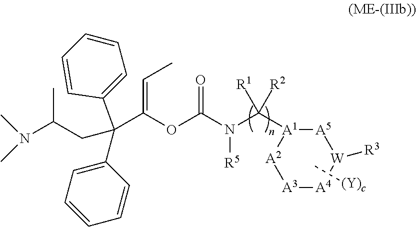

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, A⁴ and A⁵ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(IV):

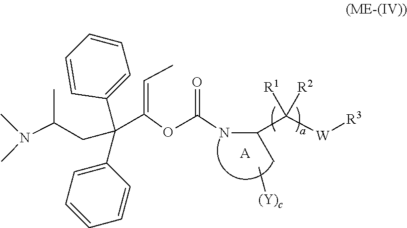

wherein:
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IVa):

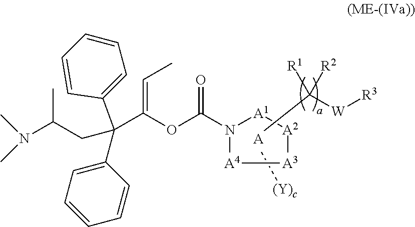

wherein:
R³ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³ and A⁴ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IVb):

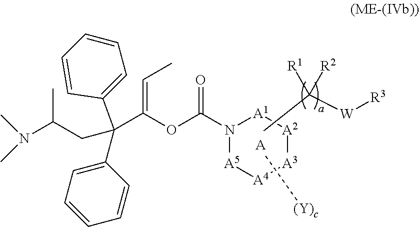

wherein:
R³ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, A⁴ and A⁵ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(IV):

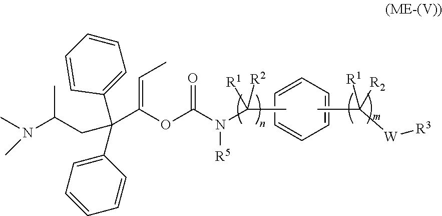

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 0 to 4;
m is an integer from 0 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The present disclosure also provides pharmaceutical compositions, and their methods of use, where the pharmaceutical compositions comprise one or more of the methadone active agents described herein.

### BRIEF DESCRIPTION OF THE FIGURES

FIGS. 1A-1B depict the concentration versus time of methadone active agents and methadone after the single 20 mg/kg oral dose. FIG. 1A depicts the concentration of Levomethadone 2R-methylethyl arginine and methadone over a 24 hour period after administration. FIG. 1B depicts the concentration of Levomethadone 1-methylethyl arginine and methadone over a 24 hour period after administration.

FIGS. 2A-2D depict dose normalized plasma and cerebrospinal fluid (CSF) concentrations of methadone and methadone active agent after a single intravenous bolus administration according to certain embodiments. FIG. 2A depicts the methadone active agent plasma concentration over a 1-hour period for administered levomethadone 1-methylethyl arginine, levomethadone 2-ethylethyl arginine, levomethadone 2R-methylethyl arginine and levomethadone butyl arginine and methadone. FIG. 2B depicts the methadone plasma concentration over a 1-hour period for administered levomethadone 1-methylethyl arginine, levomethadone 2-ethylethyl arginine, levomethadone 2R-methylethyl arginine and levomethadone butyl arginine and methadone. FIG. 2C depicts the methadone active agent CNS concentration over a 1-hour period for administered levomethadone 1-methylethyl arginine, levomethadone 2-ethylethyl arginine, levomethadone 2R-methylethyl arginine and levomethadone butyl arginine and methadone. FIG. 2D depicts the methadone CNS concentration over a 1-hour period for administered levomethadone 1-methylethyl arginine, levomethadone 2-ethylethyl arginine, levomethadone 2R-methylethyl arginine and levomethadone butyl arginine and methadone.

### DEFINITIONS

The following terms have the following meaning unless otherwise indicated. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O) R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, propionyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted hetcroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O) R⁶⁰, —C(S) R⁶⁰, —C(O)OR⁶⁰, —C(O)NR⁶⁰R⁶¹, —C(O)O⁻, —C(S) OR⁶⁰, —NR⁶²C(O)NR⁶⁰R⁶¹, —NR⁶²C(S) NR⁶⁰R⁶¹, —NR⁶²C(NR⁶³) NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O) R⁶⁰, —C(S) R⁶⁰, —C(O)OR⁶⁰, —C(O)NR⁶⁰R⁶¹, —C(O)O⁻; —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O) R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O) R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Dose unit” as used herein refers to dosage of the methadone active agent. A single dose unit provides a therapeutically effective amount of drug (i.e., a sufficient amount of drug to effect a therapeutic effect, e.g., a dose within the respective drug's therapeutic window, or therapeutic range). “Multiple dose units” or “multiples of a dose unit” or a “multiple of a dose unit” refers to at least two single dose units.

“PK profile” refers to a profile of drug concentration in blood or plasma. Such a profile can be a relationship of drug concentration over time (i.e., a “concentration-time PK profile”) or a relationship of drug concentration versus number of doses ingested (i.e., a “concentration-dose PK profile”). A PK profile is characterized by PK parameters.

“PK parameter” refers to a measure of drug concentration in blood or plasma, such as: 1) “drug Cmax”, the maximum concentration of drug achieved in blood or plasma; 2) “drug Tmax”, the time elapsed following ingestion to achieve Cmax; and 3) “drug exposure”, the total concentration of drug present in blood or plasma over a selected period of time, which can be measured using the area under the curve (AUC) of a time course of drug release over a selected period of time (t). Modification of one or more PK parameters provides for a modified PK profile.

“Pharmacodynamic (PD) profile” refers to a profile of the efficacy of a drug in a patient (or subject or user), which is characterized by PD parameters. “PD parameters” include “drug Emax” (the maximum drug efficacy), “drug EC50” (the concentration of drug at 50% of the Emax) and side effects.

“Pharmaceutical composition” refers to at least one compound and can further comprise a pharmaceutically acceptable carrier, with which the compound is administered to a patient.

“Pharmaceutically acceptable carrier” refers to a diluent, adjuvant, excipient or vehicle with, or in which a compound is administered.

“Preventing” or “prevention” or “prophylaxis” refers to a reduction in risk of occurrence of a condition, such as pain.

“Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent.

“Promoiety” refers to a form of protecting group that when used to mask a functional group within an active agent converts the active agent into a prodrug. Typically, the promoiety will be attached to the drug via bond(s) that are cleaved by enzymatic or non-enzymatic means in vivo.

“Treating” or “treatment” of any condition, such as pain, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition.

“Therapeutically effective amount” means the amount of a compound that, when administered to a patient for preventing or treating a condition such as pain, is sufficient to effect such treatment. The “therapeutically effective amount” will vary depending on the compound, the condition and its severity and the age, weight, etc., of the patient.

### DETAILED DESCRIPTION

Aspects of the present disclosure include methadone active agents, pharmaceutical compositions, and their methods of use. Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

As used herein, the singular forms “a”, “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” “consisting,” and “consists” can be used interchangeably.

The term “about” or “approximately” means within an acceptable error range for the particular value as determined by one of ordinary skill in the art, which will depend in part on how the value is measured or determined, i.e., the limitations of the measurement system. In the context of compositions containing amounts of ingredients where the terms “about” or “approximately” are used, these compositions contain the stated amount of the ingredient with a variation (error range) of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

### Methadone Active Agents

As summarized above, aspects of the present disclosure include methadone active agents.

### REPRESENTATIVE EMBODIMENTS

Reference will now be made in detail to various embodiments. It will be understood that the invention is not limited to these embodiments. To the contrary, it is intended to cover alternatives, modifications, and equivalents as may be included within the spirit and scope of the allowed claims.

In embodiments, “salts” of the compounds of the present disclosure may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. In some embodiments, salts of interest include sodium salts.

The term “solvate” as used herein refers to a complex or aggregate formed by one or more molecules of a solute, e.g. a compound of Formula ME-(I) or a salt thereof, and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments include a methadone active agent that is a compound of formula ME-(I):

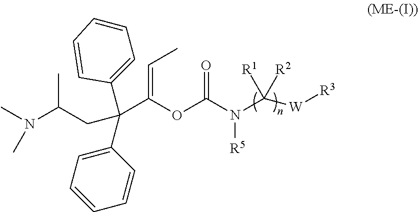

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(II):

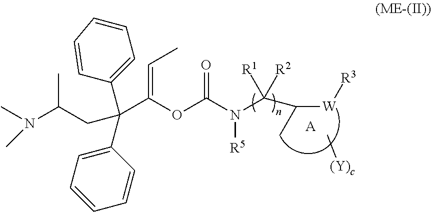

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIa):

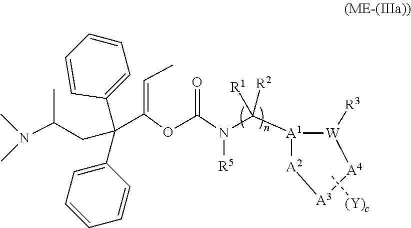

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, and A⁴ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIb):

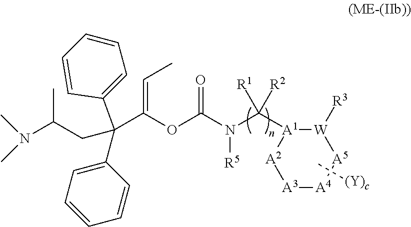

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, A⁴ and A⁵ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(III):

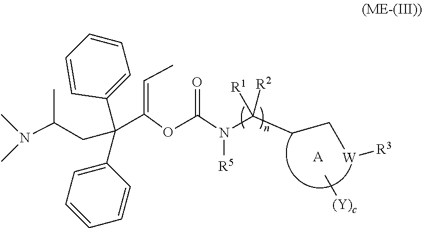

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIIa):

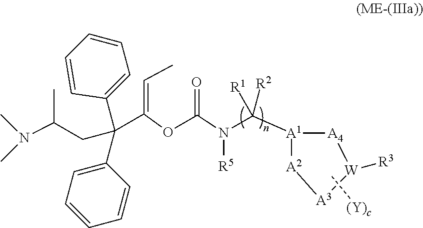

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, and A⁴ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IIIb):

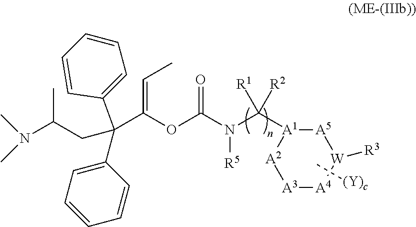

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, A⁴ and A⁵ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(IV):

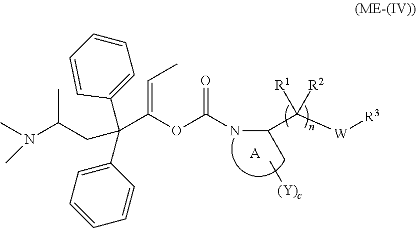

wherein:
R³ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IVa):

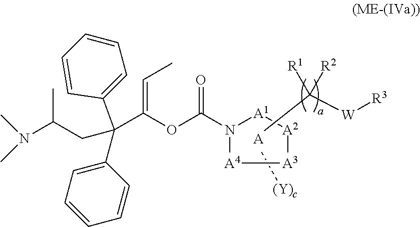

wherein:
R³ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³ and A⁴ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In certain embodiments, the methadone active agent is a compound of formulate ME-(IVb):

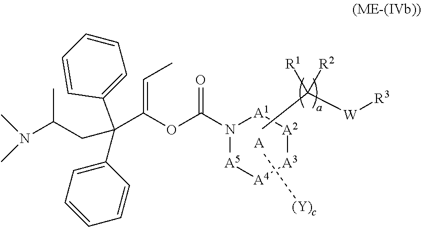

wherein:
R³ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
A¹, A², A³, A⁴ and A⁵ are independently selected from carbon, nitrogen, oxygen, and sulfur;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

The embodiments include a methadone active agent that is a compound of formula ME-(IV):

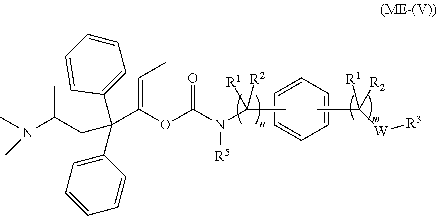

wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 0 to 4;
m is an integer from 0 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

In some instances, R³ can be selected from hydrogen, alkyl, substituted alkyl, arylalkyl, substituted arylalkyl, aryl and substituted aryl. In certain instances, R³ is hydrogen. In certain instances, R³ is (1-6C)alkyl. In other instances, R³ is (1-4C)alkyl. In some instances, R³ is selected from methyl, ethyl, n-propyl, isopropyl, n-butyl, t-butyl, pentyl and hexyl. In other instances, R³ is methyl or ethyl. In other instances, R³ is methyl. In certain instances, R³ is ethyl.

In some instances, R⁵ can be selected from hydrogen, alkyl, substituted alkyl, arylalkyl, substituted arylalkyl, aryl and substituted aryl. In certain instances, R⁵ is hydrogen. In certain instances, R⁵ is (1-6C)alkyl. In other instances, R⁵ is (1-4C)alkyl. In some instances, R⁵ is selected from methyl, ethyl, n-propyl, isopropyl, n-butyl, t-butyl, pentyl and hexyl. In other instances, R⁵ is methyl or ethyl. In other instances, R⁵ is methyl. In certain instances, R⁵ is ethyl.

In certain instances, R⁵ is substituted alkyl. In certain instances, R⁵ is an alkyl group substituted with a carboxylic group such as a carboxylic acid, carboxylic ester or carboxylic amide. In certain instances, R⁵ is —(CH₂)ₙ—COOH, —(CH₂)ₙ—COOCH₃, or —(CH₂)ₙ—COOCH₂CH₃, wherein n is a number form one to 10. In certain instances, R¹ is —(CH₂)₅—COOH), —(CH₂)₅—COOCH₃, or —(CH₂)₅—COOCH₂CH₃.

In certain instances, R⁵ is arylalkyl or substituted arylalkyl. In certain instances, R⁵ is arylalkyl. In certain instances, R⁵ is substituted arylalkyl. In certain instances, R⁵ is an arylalkyl group substituted with a carboxylic group such as a carboxylic acid, carboxylic ester or carboxylic amide. In certain instances, R⁵ is —(CH₂)_{q}(C₆H₄)—COOH, —(CH₂)_{q}(C₆H₄)—COOCH₃, or —(CH₂)_{q}(C₆H₄)—COOCH₂CH₃, where q is an integer from one to 10. In certain instances, R⁵ is —CH₂(C₆H₄)—COOH, —CH₂ (C₆H₄)—COOCH₃, or —CH₂ (C₆H₄)—COOCH₂CH₃.

In certain instances, R⁵ is aryl. In certain instances, R⁵ is substituted aryl. In certain instances, R⁵ is an aryl group ortho, meta or para-substituted with a carboxylic group such as a carboxylic acid, carboxylic ester or carboxylic amide. In certain instances, R⁵ is —(C₆H₄)—COOH, —(C₆H₄)—COOCH₃, or (C₆H₄)—COOCH₂CH₃.

In some embodiments, each R¹ can be independently selected from hydrogen, alkyl, substituted alkyl, aryl, substituted aryl, acyl, and aminoacyl. In certain instances, R¹ is hydrogen or alkyl. In certain instances, R¹ is hydrogen. In certain instances, R¹ is alkyl. In certain instances, R¹ is (1-6C)alkyl. In other instances, R¹ is (1-4C)alkyl. In some instances, R¹ is selected from methyl, ethyl, n-propyl, isopropyl, n-butyl, t-butyl, pentyl and hexyl. In other instances, R¹ is methyl or ethyl. In other instances, R¹ is methyl. In certain instances, R¹ is ethyl. In certain instances, R¹ is acyl. In certain instances, R¹ is aminoacyl.

In some embodiments, each R² can be independently selected from hydrogen, alkyl, substituted alkyl, aryl, substituted aryl, acyl, and aminoacyl. In certain instances, R² is hydrogen or alkyl. In certain instances, R² is hydrogen. In certain instances, R² is alkyl. In certain instances, R² is (1-6C)alkyl. In other instances, R² is (1-4C)alkyl. In some instances, R² is selected from methyl, ethyl, n-propyl, isopropyl, n-butyl, t-butyl, pentyl and hexyl. In other instances, R² is methyl or ethyl. In other instances, R² is methyl. In certain instances, R² is ethyl. In certain instances, R² is acyl. In certain instances, R² is aminoacyl.

In certain instances, R¹ and R² are hydrogen. In certain instances, R¹ and R² on the same carbon are both alkyl. In certain instances, R¹ and R² on the same carbon are both (1-6C)alkyl. In other instances, R¹ and R² on the same carbon are both (1-4C)alkyl. In some instances, R¹ and R² on the same carbon are both selected from methyl, ethyl, n-propyl, isopropyl, n-butyl, t-butyl, pentyl and hexyl. In certain instances, R¹ and R² on the same carbon are methyl. In certain instances, R¹ and R² on the same carbon are ethyl.

In certain instances, R¹ and R¹ which are vicinal are both alkyl and R² and R² which are vicinal are both hydrogen. In certain instances, R¹ and R¹ which are vicinal are both ethyl and R² and R² which are vicinal are both hydrogen. In certain instances, R¹ and R¹ which are vicinal are both methyl and R² and R² which are vicinal are both hydrogen.

In certain instances, in the chain of —[C(R¹)(R²)]ₙ— not every carbon is substituted. In certain instances, in the chain of —[C(R¹)(R²)]ₙ—, there is a combination of different alkyl substituents, such as methyl or ethyl.

In certain instances, one of R¹ and R² is methyl, ethyl or other alkyl and R⁵ is alkyl. In certain instances, R¹ and R¹ which are vicinal are both alkyl and R² and R² which are vicinal are both hydrogen and R⁵ is alkyl. In certain instances, R¹ and R¹ which are vicinal are both ethyl and R² and R² which are vicinal are both hydrogen and R⁵ is alkyl. In certain instances, R¹ and R¹ which are vicinal are both methyl and R² and R² which are vicinal are both hydrogen and R⁵ is alkyl.

In certain instances, one of R¹ and R² is methyl, ethyl or other alkyl and R⁵ is substituted alkyl. In certain instances, one of R¹ and R² is methyl, ethyl or other alkyl and R⁵ is an alkyl group substituted with a carboxylic group such as a carboxylic acid, carboxylic ester or carboxylic amide. In certain instances, one of R¹ and R² is methyl, ethyl or other alkyl and R⁵ is —(CH₂)_{q}(C₆H₄)—COOH, (CH₂)_{q}(C₆H₄)—COOCH₃, or —(CH₂)_{q}(C₆H₄)—COOCH₂CH₃, where q is an integer from one to 10. In certain instances, one of R¹ and R² is methyl, ethyl or other alkyl and R⁵ is an alkyl group substituted with carboxamide.

In some instances, R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group. In certain instances, R¹ and R² together with the carbon to which they are attached can form a cycloalkyl group. Thus, in certain instances, R¹ and R² on the same carbon form a spirocycle. In certain instances, R¹ and R² together with the carbon to which they are attached can form a substituted cycloalkyl group. In certain instances, two R¹ or R² groups on adjacent carbon atoms, together with the carbon atoms to which they are attached, can form a cycloalkyl group. In certain instances, two R¹ or R² groups on adjacent carbon atoms, together with the carbon atoms to which they are attached, can form a substituted cycloalkyl group.

In some embodiments, R¹ and R² together with the carbon to which they are attached can form an aryl or substituted aryl group, or two R¹ or R² groups on adjacent carbon atoms, together with the carbon atoms to which they are attached, can form an aryl or substituted aryl group. In certain instances, two R¹ or R² groups on adjacent carbon atoms, together with the carbon atoms to which they are attached, form a phenyl ring. In certain instances, two R¹ or R² groups on adjacent carbon atoms, together with the carbon atoms to which they are attached, form a substituted phenyl ring. In certain instances, two R¹ or R² groups on adjacent carbon atoms, together with the carbon atoms to which they are attached, form a naphthyl ring.

In certain instances, one of R¹ and R² is aminoacyl. In certain instances, one or both of R¹ and R² is aminoacyl comprising phenylenediamine. In certain instances, one of R¹ and R² is

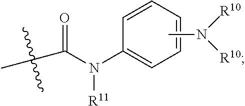

wherein each R¹⁰ is independently selected from hydrogen, alkyl, substituted alkyl, and acyl and R¹¹ is alkyl or substituted alkyl. In certain instances, at least one of R¹⁰ is acyl. In certain instances, at least one of R¹⁰ is alkyl or substituted alkyl. In certain instances, at least one of R¹⁰ is hydrogen. In certain instances, both of R¹⁰ are hydrogen.

In certain instances, one of R¹ and R²

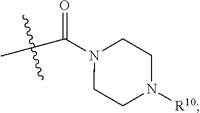

is wherein R¹⁰ is hydrogen, alkyl, substituted alkyl, or acyl. In certain instances, R¹⁰ is acyl. In certain instances, R¹⁰ is alkyl or substituted alkyl. In certain instances, R¹⁰ is hydrogen.

In certain instances, one of R¹ and R² is

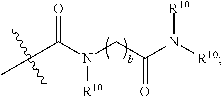

wherein each R¹⁰ is independently hydrogen, alkyl, substituted alkyl, or acyl and b is a number from one to 5. In certain instances, one of R¹ and R² is

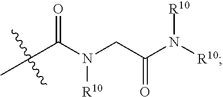

wherein each R¹⁰ is independently hydrogen, alkyl, substituted alkyl, or acyl. In certain instances, one of R¹ and R² is

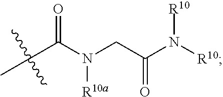

wherein R^{10a} is alkyl and each R¹⁰ is independently hydrogen, alkyl, substituted alkyl, or acyl.

In certain instances, one of R¹ and R² is

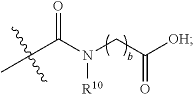

wherein R¹⁰ is independently hydrogen, alkyl, substituted alkyl, or acyl and b is a number from one to 5. In certain instances, one of R¹ and R² is

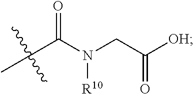

wherein R¹⁰ is independently hydrogen, alkyl, substituted alkyl, or acyl.

In certain instances, one of R¹ and R² is an aminoacyl group, such as —C(O)NR^{10a}R^{10b}, wherein each R^{10a} and R^{10b} is independently selected from hydrogen, alkyl, substituted alkyl, and acyl. In certain instances, one of R¹ and R² is an aminoacyl group, such as —C(O)NR^{10a}R^{10b}, wherein R^{10a} is an alkyl and R^{10b} is substituted alkyl. In certain instances, one of R¹ and R² is an aminoacyl group, such as —C(O)NR^{10a}R^{10b}, wherein R^{10a} is an alkyl and R^{10b} is alkyl substituted with a carboxylic acid or carboxyl ester. In certain instances, one of R¹ and R² is an aminoacyl group, such as —C(O)NR^{10a}R^{10b}, wherein R 10a is methyl and R^{10b} is alkyl substituted with a carboxylic acid or carboxyl ester.

In certain instances, R¹ or R² can modulate a rate of intramolecular cyclization. R¹ or R² can speed up a rate of intramolecular cyclization, when compared to the corresponding molecule where R¹ and R² are both hydrogen. In certain instances, R¹ or R² comprise an electron-withdrawing group or an electron-donating group. In certain instances, R¹ or R² comprise an electron-withdrawing group. In certain instances, R¹ or R² comprise an electron-donating group.

Atoms and groups capable of functioning as electron withdrawing substituents are well known in the field of organic chemistry. They include electronegative atoms and groups containing electronegative atoms. Such groups function to lower the basicity or protonation state of a nucleophilic nitrogen in the beta position via inductive withdrawal of electron density. Such groups can also be positioned on other positions along the alkylene chain. Examples include halogen atoms (for example, a fluorine atom), acyl groups (for example an alkanoyl group, an aroyl group, a carboxyl group, an alkoxycarbonyl group, an aryloxycarbonyl group or an aminocarbonyl group (such as a carbamoyl, alkylaminocarbonyl, dialkylaminocarbonyl or arylaminocarbonyl group)), an oxo (═O) substituent, a nitrile group, a nitro group, ether groups (for example an alkoxy group) and phenyl groups bearing a substituent at the ortho position, the para position or both the ortho and the para positions, each substituent being selected independently from a halogen atom, a fluoroalkyl group (such as trifluoromethyl), a nitro group, a cyano group and a carboxyl group. Each of the electron withdrawing substituents can be selected independently from these.

In certain instances, —[C(R¹)(R²)]ₙ— is selected from —CH(CH₂F)CH(CH₂F)—; —CH(CHF₂)CH(CHF₂)—; —CH(CF₃)CH(CF₃)—; —CH₂CH(CF₃)—; —CH₂CH(CHF₂)—; —CH₂CH(CH₂F)—; —CH₂CH(F)CH₂—; —CH₂C(F₂)CH₂—; —CH₂CH(C(O)NR²⁰R²¹)—; —CH₂CH(C(O)OR²²)—; —CH₂CH(C(O)OH)—; —CH(CH₂F)CH₂CH(CH₂F)—; —CH(CHF₂)CH₂CH(CHF₂)—; —CH(CF₃)CH₂CH(CF₃)—; —CH₂CH₂CH(CF₃)—; —CH₂CH₂CH(CHF₂)—; —CH₂CH₂CH(CH₂F)—; —CH₂CH₂CH(C(O)NR²³R²⁴)—; —CH₂CH₂CH(C(O)OR²⁵)—; and —CH₂CH₂CH(C(O)OH)—, in which R²⁰, R²¹, R²² and R²³ each independently represents hydrogen or (1-6C)alkyl, and R²⁴ and R²⁵ each independently represents (1-6C)alkyl.

In methadone active agent compounds according to embodiments of the present disclosure, n can be an integer from 1 to 10. In certain instances, n is one. In certain instances, n is two. In other instances, n is three. In other instances, n is four. In certain instances, n is five.

In methadone active agent compounds according to embodiments of the present disclosure, a can be an integer from 1 to 10. In certain instances, a is one. In certain instances, a is two. In other instances, a is three. In other instances, a is four. In certain instances, a is five.

In methadone active agent compounds according to embodiments of the present disclosure, a can be an integer from 1 to 10. In certain instances, m is one. In certain instances, m is two. In other instances, m is three. In other instances, m is four. In certain instances, m is five.

In methadone active agent compounds according to embodiments of the present disclosure, c can be an integer from 1 to 10. In certain instances, c is one. In certain instances, c is two. In other instances, c is three. In other instances, c is four. In certain instances, c is five.

In some embodiments, the A ring is a heterocyclic 5 to 12-membered ring. In some instances, the A ring is a 5-membered ring. In some instances, the A ring is a 6-membered ring. In certain instances, the A ring is a 5-membered nitrogen-containing ring. In certain instances, the A ring is a 5-membered oxygen-containing ring. In certain instances, the A ring is a 5-membered sulfur-containing ring. In certain instances, the A ring is a 6-membered nitrogen-containing ring. In certain instances, the A ring is a 6-membered oxygen-containing ring. In certain instances, the A ring is a 6-membered sulfur-containing ring.

In some embodiments, A¹, A², A⁴, and A⁵ are independently selected from carbon, nitrogen, oxygen, and sulfur. In some instances, A¹ is carbon. In some instances, A¹ is oxygen. In some instances, A¹ is nitrogen. In some instances, A¹ is sulfur. In some instances, A² is carbon. In some instances, A² is oxygen. In some instances, A² is nitrogen. In some instances, A² is sulfur. In some instances, A³ is carbon. In some instances, A³ is oxygen. In some instances, A³ is nitrogen. In some instances, A³ is sulfur. In some instances, A⁴ is carbon. In some instances, A⁴ is oxygen. In some instances, A⁴ is nitrogen. In some instances, A⁴ is sulfur. In some instances, A⁵ is carbon. In some instances, A⁵ is oxygen. In some instances, A⁵ is nitrogen. In some instances, A⁵ is sulfur.

In some instances, the chain of —[C(R¹)(R²)]ₐ— is substituted at A¹. In some instances, the chain of —[C(R¹)(R²)]ₐ— is substituted at A². In some instances, the chain of —[C(R¹)(R²)]ₐ— is substituted at A³. In some instances, the chain of —[C(R¹)(R²)]ₐ— is substituted at A⁴. In some instances, the chain of —[C(R¹)(R²)]ₐ— is substituted at A⁵.

In some instances, Y is alkyl. In some instances, Y is methyl. In some instances, Y is gem-dimethyl. In some instances, Y is alkoxycarbonyl. In some instances, Y is methoxycarbonyl.

In some instances, the chain of —[C(R¹)(R²)]ₙ— is substituted ortho to the chain of —[C(R¹)(R²)]ₘ—. In some instances, the chain of —[C(R¹)(R²)]ₙ— is substituted meta to the chain of —[C(R¹)(R²)]ₘ—. In some instances, the chain of —[C(R¹)(R²)]ₙ— is substituted para to the chain of —[C(R¹)(R²)]ₘ—.

In certain embodiments, the methadone active agent is Compound ME-101:

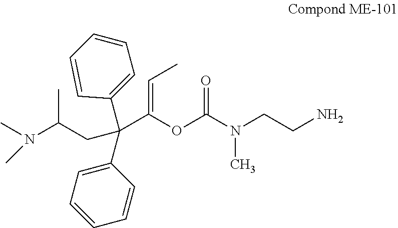 [SMILES: C/C=C(\OC(=O)N(C)CCN)C(CC(C)N(C)C)(c1ccccc1)c1ccccc1]

or a salt, hydrate or solvate thereof.

In certain embodiments, the methadone active agent is Compound ME-102:

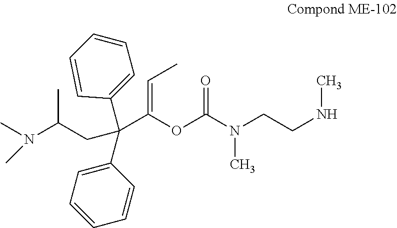

or a salt, hydrate or solvate thereof.

In certain embodiments, the methadone active agent is Compound ME-103:

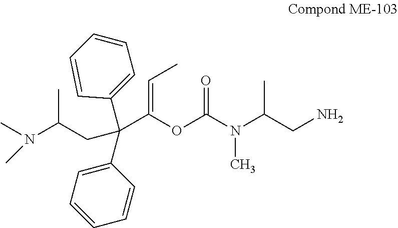 [SMILES: C/C=C(\OC(=O)N(C)C(C)CN)C(CC(C)N(C)C)(c1ccccc1)c1ccccc1]

or a salt, hydrate or solvate thereof.

In certain embodiments, the methadone active agent is Compound ME-104:

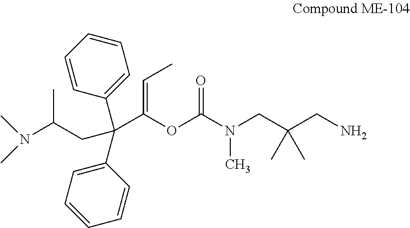 [SMILES: C/C=C(\OC(=O)N(C)CC(C)(C)CN)C(CC(C)N(C)C)(c1ccccc1)c1ccccc1]

or a salt, hydrate or solvate thereof.

In certain embodiments, the methadone active agent is Compound ME-105:

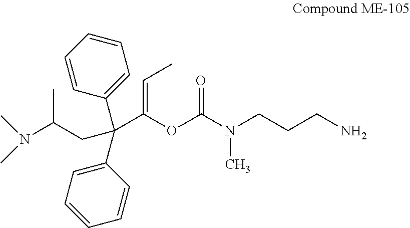 [SMILES: C/C=C(\OC(=O)N(C)CCCN)C(CC(C)N(C)C)(c1ccccc1)c1ccccc1]

or a salt, hydrate or solvate thereof.

In certain embodiments, the methadone active agent is Compound ME-106:

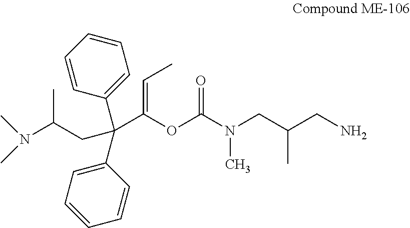 [SMILES: C/C=C(\OC(=O)N(C)CC(C)CN)C(CC(C)N(C)C)(c1ccccc1)c1ccccc1]

or a salt, hydrate or solvate thereof.

In certain embodiments, the methadone active agent is Compound ME-107:

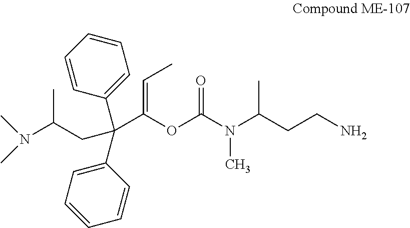 [SMILES: C/C=C(\OC(=O)N(C)C(C)CCN)C(CC(C)N(C)C)(c1ccccc1)c1ccccc1]

or a salt, hydrate or solvate thereof.

In certain embodiments, the methadone active agent is Compound ME-108:

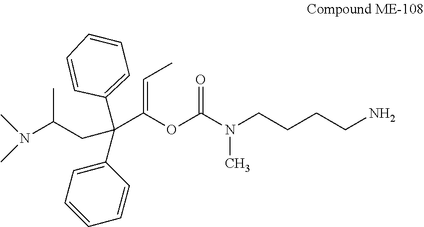 [SMILES: C/C=C(\OC(=O)N(C)CCCCN)C(CC(C)N(C)C)(c1ccccc1)c1ccccc1]

or a salt, hydrate or solvate thereof.

In certain embodiments, the methadone active agent is Compound ME-109:

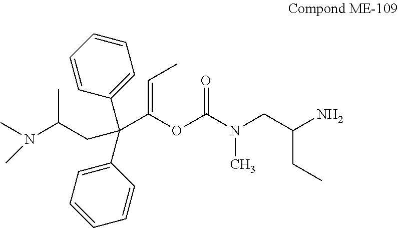 [SMILES: C/C=C(\OC(=O)N(C)CC(N)CC)C(CC(C)N(C)C)(c1ccccc1)c1ccccc1]

or a salt, hydrate or solvate thereof.

In certain embodiments, the methadone active agent is Compound ME-110:

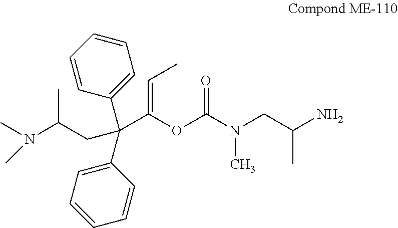 [SMILES: C/C=C(\OC(=O)N(C)CC(C)N)C(CC(C)N(C)C)(c1ccccc1)c1ccccc1]

or a salt, hydrate or solvate thereof.

In certain embodiments, the methadone active agent is Compound ME-111:

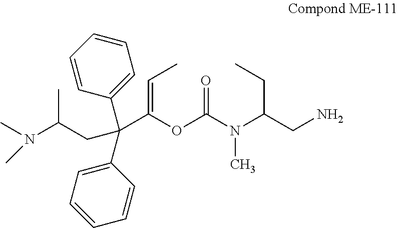 [SMILES: C/C=C(\OC(=O)N(C)C(CC)CN)C(CC(C)N(C)C)(c1ccccc1)c1ccccc1]

or a salt, hydrate or solvate thereof.

In certain embodiments, the methadone active agent is Compound ME-112:

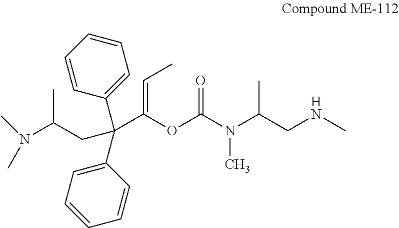

or a salt, hydrate or solvate thereof.

In certain embodiments, the methadone active agent is Compound ME-113:

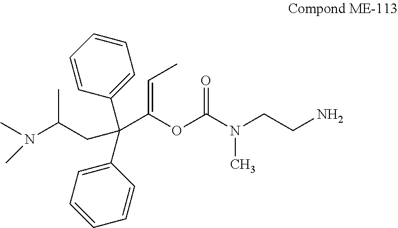 [SMILES: C/C=C(\OC(=O)N(C)CCN)C(CC(C)N(C)C)(c1ccccc1)c1ccccc1]

or a salt, hydrate or solvate thereof.

In certain embodiments, the methadone active agent is Compound ME-114:

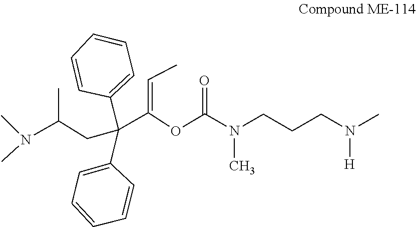

or a salt, hydrate or solvate thereof.

In certain embodiments, the methadone active agent is Compound ME-115:

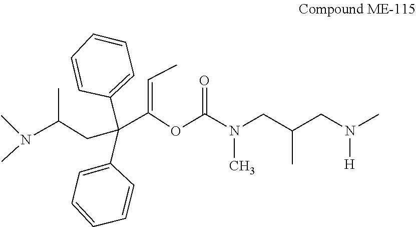

or a salt, hydrate or solvate thereof.

In certain embodiments, the methadone active agent is Compound ME-116:

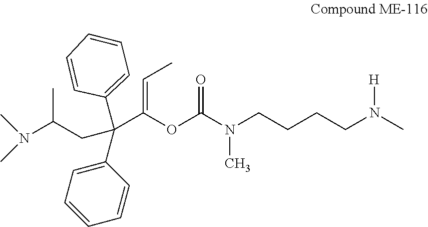

or a salt, hydrate or solvate thereof.

In certain embodiments, the methadone active agent is Compound ME-117:

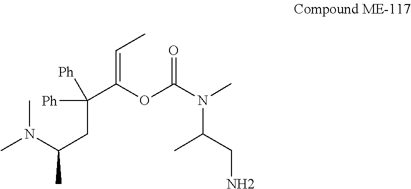 [SMILES: C/C=C(\OC(=O)N(C)C(C)CN)C(C[C@@H](C)N(C)C)(c1ccccc1)c1ccccc1]

or a salt, hydrate or solvate thereof.

In certain embodiments, the methadone active agent is Compound ME-118:

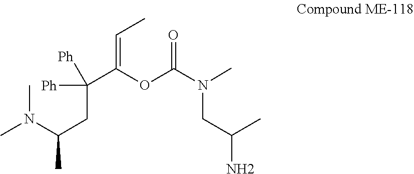 [SMILES: C/C=C(\OC(=O)N(C)CC(C)N)C(C[C@@H](C)N(C)C)(c1ccccc1)c1ccccc1]

or a salt, hydrate or solvate thereof.

In certain embodiments, the methadone active agent is Compound ME-119:

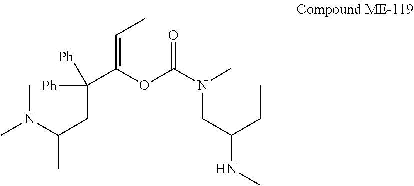 [SMILES: C/C=C(\OC(=O)N(C)CC(CC)NC)C(CC(C)N(C)C)(c1ccccc1)c1ccccc1]

or a salt, hydrate or solvate thereof.

In certain embodiments, the methadone active agent is Compound ME-120:

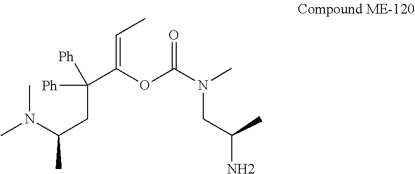 [SMILES: C/C=C(\OC(=O)N(C)C[C@@H](C)N)C(C[C@@H](C)N(C)C)(c1ccccc1)c1ccccc1]

or a salt, hydrate or solvate thereof.

In certain embodiments, the methadone active agent is Compound ME-121:

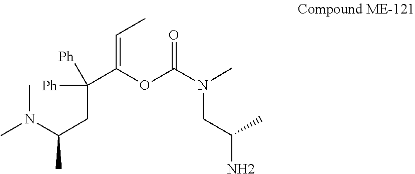 [SMILES: C/C=C(\OC(=O)N(C)C[C@H](C)N)C(C[C@@H](C)N(C)C)(c1ccccc1)c1ccccc1]

or a salt, hydrate or solvate thereof.

In certain embodiments, the methadone active agent is Compound ME-122:

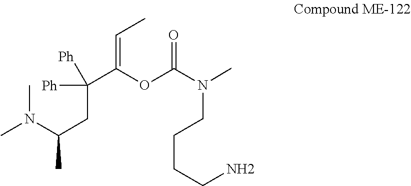 [SMILES: C/C=C(\OC(=O)N(C)CCCCN)C(C[C@@H](C)N(C)C)(c1ccccc1)c1ccccc1]

or a salt, hydrate or solvate thereof.

In certain embodiments, the methadone active agent is (R, E)-6-(dimethylamino)-4,4-diphenylhept-2-en-3-yl (1-aminopropan-2-yl)/methyl)carbamate.

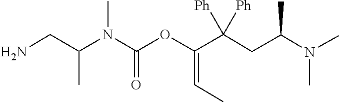 [SMILES: C/C=C(/OC(=O)N(C)C(C)CN)C(C[C@@H](C)N(C)C)(c1ccccc1)c1ccccc1]

or a salt, hydrate or solvate thereof.

In certain embodiments, the methadone active agent is (R, E)-6-(dimethylamino)-4,4-diphenylhept-2-en-3-yl ((R)-2-aminopropyl)(methyl)carbamate:

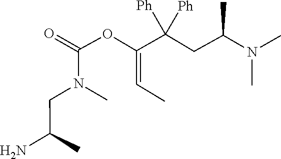 [SMILES: C/C=C(/OC(=O)N(C)C[C@@H](C)N)C(C[C@@H](C)N(C)C)(c1ccccc1)c1ccccc1]

or a salt, hydrate or solvate thereof.

In certain embodiments, the methadone active agent is (R, E)-6-(dimethylamino)-4,4-diphenylhept-2-en-3-yl ((S)-2-aminopropyl)(methyl)carbamate:

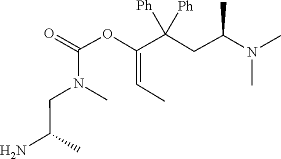 [SMILES: C/C=C(/OC(=O)N(C)C[C@H](C)N)C(C[C@@H](C)N(C)C)(c1ccccc1)c1ccccc1]

or a salt, hydrate or solvate thereof.

In certain embodiments, the methadone active agent is (R, E)-6-(dimethylamino)-4,4-diphenylhept-2-en-3-yl (4-aminobutyl)(methyl)carbamate:

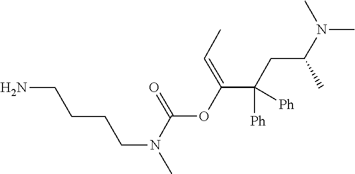 [SMILES: C/C=C(/OC(=O)N(C)CCCCN)C(C[C@@H](C)N(C)C)(c1ccccc1)c1ccccc1]

or a salt, hydrate or solvate thereof.

In certain embodiments, the methadone active agent is (R,E)-6-(dimethylamino)-4,4-diphenylhept-2-en-3-yl(2-aminobutyl)(methyl)carbamate:

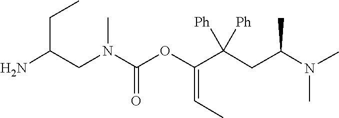 [SMILES: C/C=C(/OC(=O)N(C)CC(N)CC)C(C[C@@H](C)N(C)C)(c1ccccc1)c1ccccc1]

or a salt, hydrate or solvate thereof.

In certain embodiments, the methadone active agent is Compound ME-301:

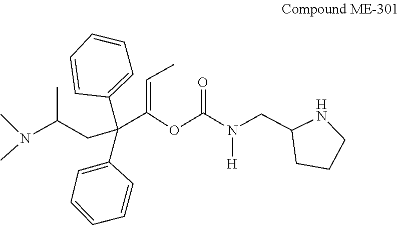 [SMILES: C/C=C(\OC(=O)NCC1CCCN1)C(CC(C)N(C)C)(c1ccccc1)c1ccccc1]

or a salt, hydrate or solvate thereof.

In certain embodiments, the methadone active agent is Compound ME-302:

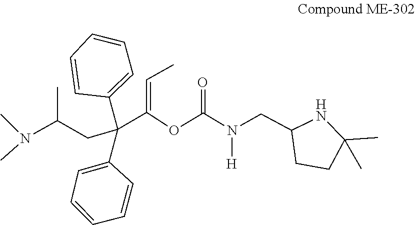

or a salt, hydrate or solvate thereof.

In certain embodiments, the methadone active agent is Compound ME-303:

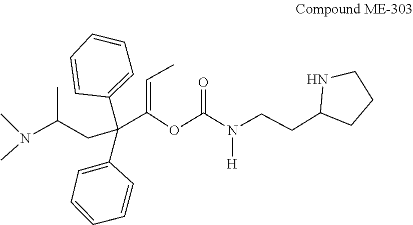 [SMILES: C/C=C(\OC(=O)NCCC1CCCN1)C(CC(C)N(C)C)(c1ccccc1)c1ccccc1]

or a salt, hydrate or solvate thereof.

In certain embodiments, the methadone active agent is Compound 304:

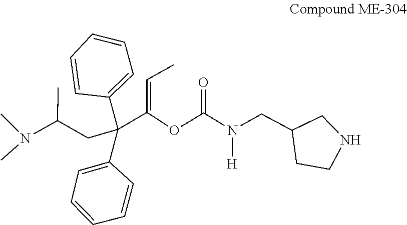 [SMILES: C/C=C(\OC(=O)NCC1CCNC1)C(CC(C)N(C)C)(c1ccccc1)c1ccccc1]

or a salt, hydrate or solvate thereof.

In certain embodiments, the methadone active agent is Compound ME-305:

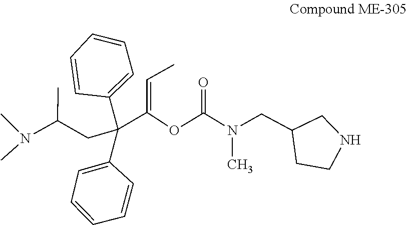 [SMILES: C/C=C(\OC(=O)N(C)CC1CCNC1)C(CC(C)N(C)C)(c1ccccc1)c1ccccc1]

or a salt, hydrate or solvate thereof.

In certain embodiments, the methadone active agent is Compound ME-306:

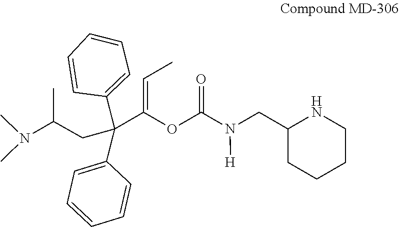 [SMILES: C/C=C(\OC(=O)NCC1CCCCN1)C(CC(C)N(C)C)(c1ccccc1)c1ccccc1]

or a salt, hydrate or solvate thereof.

In certain embodiments, the methadone active agent is Compound ME-307:

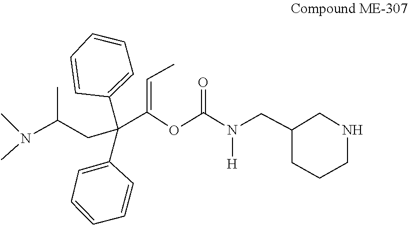 [SMILES: C/C=C(\OC(=O)NCC1CCCNC1)C(CC(C)N(C)C)(c1ccccc1)c1ccccc1]

or a salt, hydrate or solvate thereof.

In certain embodiments, the methadone active agent is Compound ME-308:

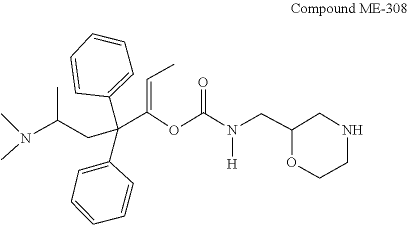 [SMILES: C/C=C(\OC(=O)NCC1CNCCO1)C(CC(C)N(C)C)(c1ccccc1)c1ccccc1]

or a salt, hydrate or solvate thereof.

In certain embodiments, the methadone active agent is Compound ME-309:

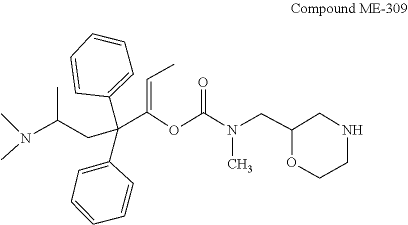 [SMILES: C/C=C(\OC(=O)N(C)CC1CNCCO1)C(CC(C)N(C)C)(c1ccccc1)c1ccccc1]

or a salt, hydrate or solvate thereof.

In certain embodiments, the methadone active agent is Compound ME-310:

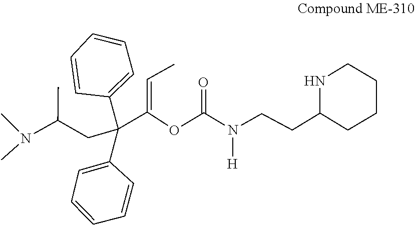 [SMILES: C/C=C(\OC(=O)NCCC1CCCCN1)C(CC(C)N(C)C)(c1ccccc1)c1ccccc1]

or a salt, hydrate or solvate thereof.

In certain embodiments, the methadone active agent is Compound ME-401:

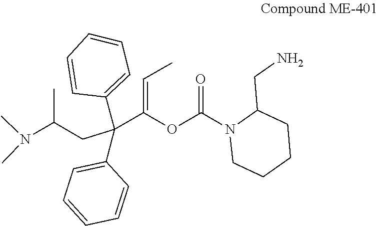 [SMILES: C/C=C(\OC(=O)N1CCCCC1CN)C(CC(C)N(C)C)(c1ccccc1)c1ccccc1]

or a salt, hydrate or solvate thereof.

In certain embodiments, the methadone active agent is Compound ME-402:

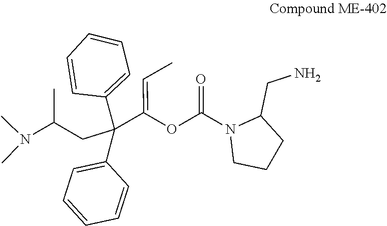 [SMILES: C/C=C(\OC(=O)N1CCCC1CN)C(CC(C)N(C)C)(c1ccccc1)c1ccccc1]

or a salt, hydrate or solvate thereof.

In certain embodiments, the methadone active agent is Compound ME-403:

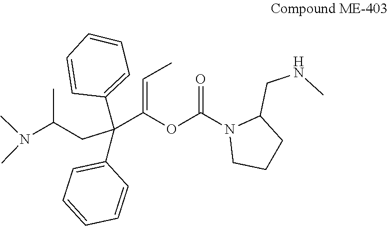

or a salt, hydrate or solvate thereof.

In certain embodiments, the methadone active agent is Compound ME-404:

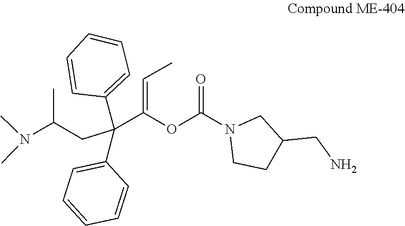 [SMILES: C/C=C(\OC(=O)N1CCC(CN)C1)C(CC(C)N(C)C)(c1ccccc1)c1ccccc1]

or a salt, hydrate or solvate thereof.

In certain embodiments, the methadone active agent is Compound ME-405:

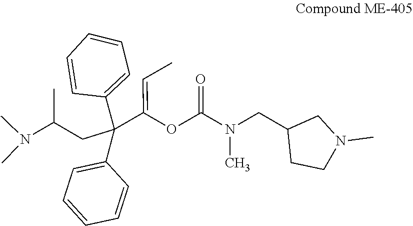

or a salt, hydrate or solvate thereof.

In certain embodiments, the methadone active agent is Compound ME-406:

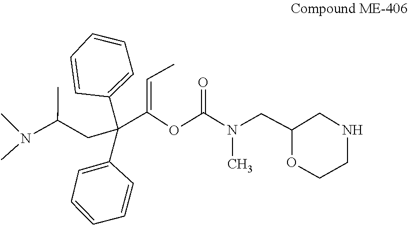 [SMILES: C/C=C(\OC(=O)N(C)CC1CNCCO1)C(CC(C)N(C)C)(c1ccccc1)c1ccccc1]

or a salt, hydrate or solvate thereof.

In certain embodiments, the methadone active agent is Compound ME-501:

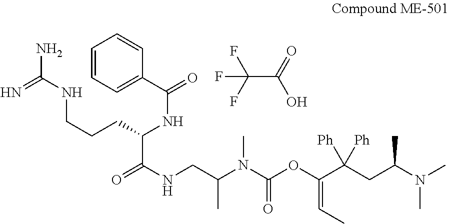

or a salt, hydrate or solvate thereof.

In certain embodiments, the methadone active agent is Compound ME-502:

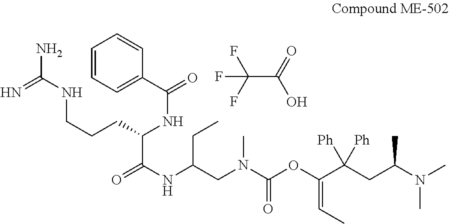

or a salt, hydrate or solvate thereof.

In certain embodiments, the methadone active agent is Compound ME-503:

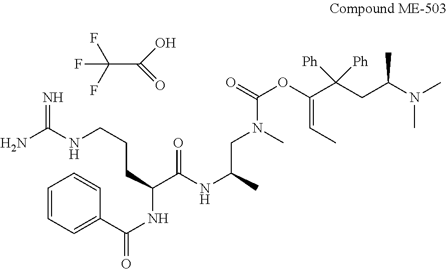

or a salt, hydrate or solvate thereof.

In certain embodiments, the methadone active agent is Compound ME-504:

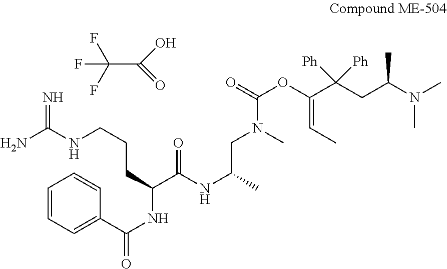

or a salt, hydrate or solvate thereof.

In certain embodiments, the methadone active agent is Compound ME-505:

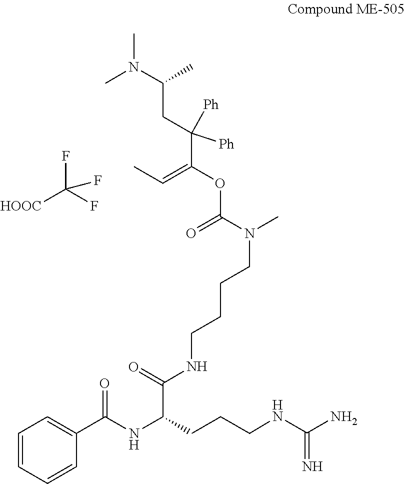

or a salt, hydrate or solvate thereof.

In some embodiments, the methadone active agents are formulated in any convenient form suitable for oral (including buccal and sublingual) administration for example as a tablet, capsule, powder, suspension, dispersion or emulsion. Pharmaceutical compositions of the methadone active agents may include one or more pharmaceutically acceptable carriers. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The amount of methadone active agent in a unit composition, for example, a capsule or tablet of the methadone active agent or pharmaceutically acceptable salt thereof, may include from 1 mg and 400 mg of methadone active agent or pharmaceutically acceptable salt thereof, for example, between: 1 and 10 mg, 10 and 20 mg, 20 and 30 mg, 30 and 40 mg, 40 and 50 mg, 50 and 60 mg, 60 and 70 mg, 70 and 80 mg, 80 and 90 mg, 90 and 100 mg, 100 and 110 mg, 110 and 120 mg, 120 and 130 mg, 130 and 140 mg, 140 and 150 mg, 150 and 160 mg, 160 and 170 mg, 170 and 180 mg, 180 and 190 mg, 190 and 200 mg, 200 and 210 mg, 210 and 220 mg, 220 and 230 mg, 230 and 240 mg, 240 and 250 mg, 250 and 260 mg, 260 and 270 mg, 270 and 280 mg, 280 and 290 mg, 290 and 300 mg, 300 and 310 mg, 310 and 320 mg, 320 and 330 mg, 330 and 340 mg, 340 and 350 mg, 350 and 360 mg, 360 and 370 mg, 370 and 380 mg, 380 and 390 mg and between 390 and 400 mg.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, pharmaceutical compositions of the methadone active agent include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Pharmaceutical Compositions and Methods of Use

As disclosed herein, the embodiments provide a composition, which comprises a methadone active agent as described herein. The pharmaceutical composition according to the embodiments can further comprise a pharmaceutically acceptable carrier. The composition is conveniently formulated as described above in a form suitable for oral (including buccal and sublingual) administration, for example as a tablet, capsule, thin film, powder, suspension, solution, syrup, dispersion or emulsion. The composition can contain components conventional in pharmaceutical preparations, e.g. one or more carriers, binders, lubricants, excipients (e.g., to impart controlled release characteristics), pH modifiers, sweeteners, bulking agents, coloring agents or further active agents.

Patients can be humans, and also other mammals, such as livestock, zoo animals and companion animals, such as a cat, dog or horse.

In another aspect, the embodiments provide a pharmaceutical composition as described hereinabove for use in the treatment of pain. The pharmaceutical composition according to the embodiments is useful, for example, in the treatment of a patient suffering from, or at risk of suffering from, pain. Accordingly, the present disclosure provides methods of treating or preventing pain in a subject, the methods involving administering to the subject a disclosed composition. The present disclosure provides for a disclosed composition for use in therapy or prevention or as a medicament. The present disclosure also provides the use of a disclosed composition for the manufacture of a medicament, especially for the manufacture of a medicament for the treatment or prevention of pain.

The compositions of the present disclosure can be used in the treatment or prevention of pain including, but not limited to include, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain.

The present disclosure also provides use of a methadone active agent as described herein in the treatment of pain. The present disclosure also provides use of a methadone active agent as described herein in the prevention of pain.

The present disclosure provides use of a methadone active agent as described herein in the manufacture of a medicament for treatment of pain. The present disclosure provides use of a methadone active agent as described herein in the manufacture of a medicament for prevention of pain.

In another aspect, the embodiments provide a method of treating pain in a patient in need thereof, which comprises administering an effective amount of a pharmaceutical composition as described hereinabove. In another aspect, the embodiments provide a method of preventing pain in a patient in need thereof, which comprises administering an effective amount of a pharmaceutical composition as described hereinabove.

The amount of composition disclosed herein to be administered to a patient to be effective (i.e. to provide blood levels of the methadone active agent sufficient to be effective in the treatment or prophylaxis of pain) will depend upon the bioavailability of the particular composition, the susceptibility of the particular composition to enzyme degradation in the gut, as well as other factors, such as the species, age, weight, sex, and condition of the patient, manner of administration and judgment of the prescribing physician. In general, the composition dose can be such that the methadone active agent is in the range of from 0.01 milligrams active agent per kilogram to 20 milligrams active agent per kilogram (mg/kg) body weight. For example, a composition comprising a methadone active agent as described herein can be administered at a dose equivalent to administering free methadone in the range of from 0.02 to 0.5 mg/kg body weight or 0.01 mg/kg to 10 mg/kg body weight or 0.01 to 2 mg/kg body weight. In one embodiment, the composition can be administered at a dose such that the level of methadone active agent achieved in the blood is in the range of from 0.5 ng/ml to 10 ng/ml.

### Methods for Administering a Methadone Active Agent or Pharmaceutically Acceptable Salt Thereof

Aspects of the present disclosure also include methods for administering to a subject a methadone active agent or a pharmaceutically acceptable salt thereof. In practicing the subject methods according to certain embodiments, one or more doses of the methadone active agents described herein are orally (including buccally or sublingually) administered to the subject. The desired protocol used to administer the methadone active agent and the appropriate dosage as described herein may, in certain embodiments, be determined by a qualified healthcare professional (e.g., a physician).

In some instances, the methadone active agent is administered simultaneously with one or more other active agents. Where the methadone active agent is administered simultaneously with another active agent, the methadone active agent may be administered as a separate composition (e.g., as a pharmaceutical composition that contains the methadone active agent and one or more pharmaceutically acceptable excipients) or may be co-formulated with the other active agent. Where the methadone active agent is co-formulated with the other active agent, the two components may be combined in a capsule. In some instances, the two components (i.e., methadone active agent and other active agent) are co-mixed within the capsule. In other instances, the two components are separated within the capsule, such as with a barrier (e.g., a water soluble membrane).

In some cases, the methadone active agent and the other active agent are administered sequentially. In some cases, the other active agent is orally administered to the subject a predetermined period of time before administering the methadone active agent. For example, the other active agent may be orally administered to the subject 1 minute or more before administering the methadone active agent, such as 2 minutes or more, such as 3 minutes or more, such as 4 minutes or more, such as 5 minutes or more, such as 10 minutes or more, such as 15 minutes or more, such as 30 minutes or more, such as 45 minutes or more, such as 60 minutes or more, such as 2 hours or more, such as 3 hours or more, such as 4 hours or more, such as 6 hours or more, such as 8 hours or more, such as 12 hours or more, such as 16 hours or more, such as 20 hours or more and including administering the other active agent may be orally administered to the subject 24 hours or more before administering the methadone active agent.

In some cases, the other active agent is orally administered to the subject a predetermined period of time after administering the methadone active agent. For example, the other active agent may be orally administered to the subject 1 minute or more after administering the methadone active agent, such as 2 minutes or more, such as 3 minutes or more, such as 4 minutes or more, such as 5 minutes or more, such as 10 minutes or more, such as 15 minutes or more, such as 30 minutes or more, such as 45 minutes or more, such as 60 minutes or more, such as 2 hours or more, such as 3 hours or more, such as 4 hours or more, such as 6 hours or more, such as 8 hours or more, such as 12 hours or more, such as 16 hours or more, such as 20 hours or more and including administering the other active agent to the subject 24 hours or more after administering the methadone active agent.

The dosage amount of the methadone active agent administered to the subject may vary, ranging from about 0.1 mg/kg to 200 mg/kg per day, such as from 0.5 mg/kg to 100 mg/kg per day, such as 1.0 mg/kg to 50 mg/kg per day, such as 2 mg/kg to 40 mg/kg per day, such as 5 mg/kg to 30 mg/kg per day, and including 10 mg/kg to 20 mg/kg per day. In embodiments, the methadone active agent may be administered to the subject once per day, twice per day, three times per day, four times per day, five times per day or at some other interval. In one embodiment the methadone active agent is administered at a dose such that the level of the active agent achieved in the blood is in the range of from 0.001 ng/ml to 500 ng/ml, such as from 0.005 ng/ml to 450 ng/ml, such as from 0.01 ng/ml to 400 ng/ml, such as from 0.05 ng/ml to 350 ng/ml, such as from 0.1 ng/ml to 300 ng/ml, such as from 0.5 ng/ml to 250 ng/ml, such as from 1 ng/ml to 200 ng/ml, such as from 1.5 ng/ml to 100 ng/ml, such as from 2 ng/ml to 50 ng/ml and including from 3 ng/ml to 25 ng/ml.

Each treatment interval with the methadone active agent may be 1 day or longer, such as 2 days or longer, such as 3 days or longer, such as 4 days or longer, such as 5 days or longer, such as 6 days or longer, such as 7 days or longer, such as 2 weeks or longer, such as 3 weeks or longer, such as 4 weeks or longer, such as 8 weeks or longer, such as 12 weeks or longer, such as 16 weeks or longer, such as 20 weeks or longer, such as 24 weeks or longer, such as 28 weeks or longer, such as 32 weeks or longer, such as 36 weeks or longer, such as 40 weeks or longer, such as 44 weeks or longer, such as 48 weeks or longer and including 52 weeks or longer. In certain embodiments, protocols may include multiple dosage intervals. In practicing methods of the present disclosure, treatment regimens may include two or more dosage intervals, such as three or more dosage intervals, such as four or more dosage intervals, such as five or more dosage intervals, including ten or more dosage intervals.

In some instances, the methadone active agent is administered to the subject once or more per day in a cycle for a duration of 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days or 2 days or 1 day. In some instances, the methadone active agent is administered to the subject once per day for a duration of from about 1 day to about 30 days, such as once per day for a duration of from about 1 day to about 28 days, from 1 day to 21 days, from 7 days to 14 days. In certain instances, the methadone active agent is administered to the subject once per day for a duration of 14 days. In other instances, the methadone active agent is administered to the subject twice per day for a duration of from about 1 day to about 30 days, such as once per day for a duration of from about 1 day to about 28 days, from 1 day to 21 days, from 7 days to 14 days. In certain instances, the methadone active agent is administered to the subject twice per day for a duration of 14 days.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

In some embodiments, dosing is administered in cycles of administration of methadone active agent. In some embodiments, the cycle is 21 days or more, in some instances the cycle is 28 days or more. The cycles of drug administration may be repeated for 1, 2, 3, 4, 5, 6, 7, 8 or more than 8 dosage cycles, for a total period of 6 months or 1 year or 2 years or 3 years or 4 years or more. This administration cycle may be repeated, such as 2 or more times, such as 3 or more times, such as 4 or more times, such as 5 or more times, such as 6 or more time, such as 7 or more times, such as 8 or more times, such as 9 or more times and including 10 or more times.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Oral Administration

### Pharmacokinetic Study of Methadone Active Agents Following Oral Gavage Administration in Rats

This study evaluated 4 compounds at 20 mg/kg for plasma PK of methadone active agents and methadone following oral administration. The methadone active agents included:

### 1) Levomethadone 1-methylethyl arginine (Levo-1ME)

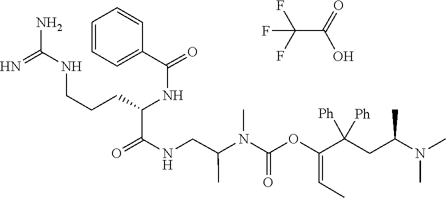 [SMILES: C/C=C(/OC(=O)N(C)C(C)CNC(=O)[C@H](CCCNC(=N)N)NC(=O)c1ccccc1)C(C[C@@H](C)N(C)C)(c1ccccc1)c1ccccc1 ; O=C(O)C(F)(F)F]

### (R)-6-(dimethylamino)-4,4-diphenylhept-2-en-3-yl (1-((S)-2-benzamido-5-guanidinopentanamido)propan-2-yl)(methyl)carbamate 2,2,2-trifluoroacetate

### 2) Levomethadone 2-ethylethyl arginine (Levo-2EE)

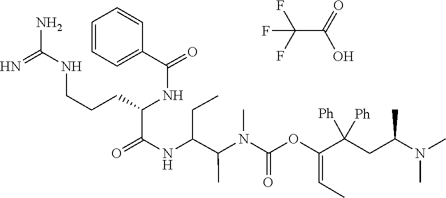 [SMILES: C/C=C(/OC(=O)N(C)C(C)C(CC)NC(=O)[C@H](CCCNC(=N)N)NC(=O)c1ccccc1)C(C[C@@H](C)N(C)C)(c1ccccc1)c1ccccc1 ; O=C(O)C(F)(F)F]

### 6-(dimethylamino)-4,4-diphenylhept-2-en-3-yl(2-((S)-2-benzamido-5-guanidinopentanamido)butyl)(methyl)carbamate 2,2,2-trifluoroacetate

### 3) Levomethadone 2R-methylethyl arginine (Levo-2R-ME)

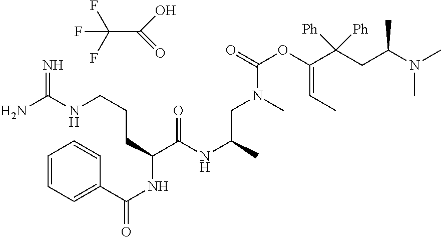

### (R)-6-(dimethylamino)-4,4-diphenylhept-2-en-3-yl ((R)-2-((S)-2-benzamido-5-guanidinopentanamido) propyl)(methyl)carbamate 2,2,2-trifluoroacetate

### 4) Levomethadone 2S-methylethyl arginine (Levo-2S-ME)

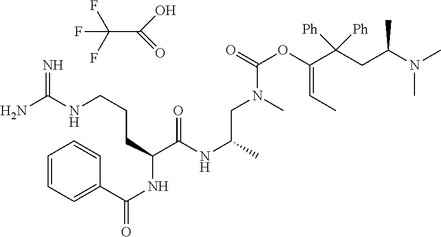

### (R)-6-(dimethylamino)-4,4-diphenylhept-2-en-3-yl ((S)-2-((S)-2-benzamido-5-guanidinopentanamido) propyl)(methyl)carbamate 2,2,2-trifluoroacetate

### 5) Levomethadone butyl arginine (Levo-Butyl)

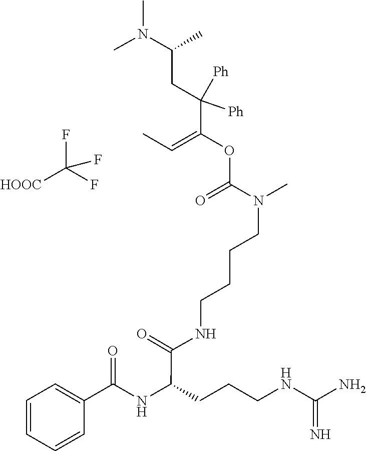 [SMILES: C/C=C(/OC(=O)N(C)CCCCNC(=O)[C@H](CCCNC(=N)N)NC(=O)c1ccccc1)C(C[C@@H](C)N(C)C)(c1ccccc1)c1ccccc1 ; O=C(O)C(F)(F)F]

### (R)-6-(dimethylamino)-4,4-diphenylhept-2-en-3-yl (4-((S)-2-benzamido-5-guanidinopentanamido)butyl)(methyl)carbamate 2,2,2-trifluoroacetate

Each of the compounds (Levo 1-ME, Levo 2R-ME, Levo 2EE, and Levo-Butyl) was administered by a single 20 mg/kg oral gavage (PO) administration in jugular vein-cannulated rats (3/group). PK plasma samples were collected predose and at 0.25, 0.5, 1, 2, 4, 6, 8, 12, and 24 hours postdose and shipped for determination of parent active agent and methadone concentrations. The qualitative presence or absence of a peak representing the intermediate form of each active agent after removal of the amino acid tail was also assessed. Due to the lack of an intermediate reference standard, this peak could not be quantified.

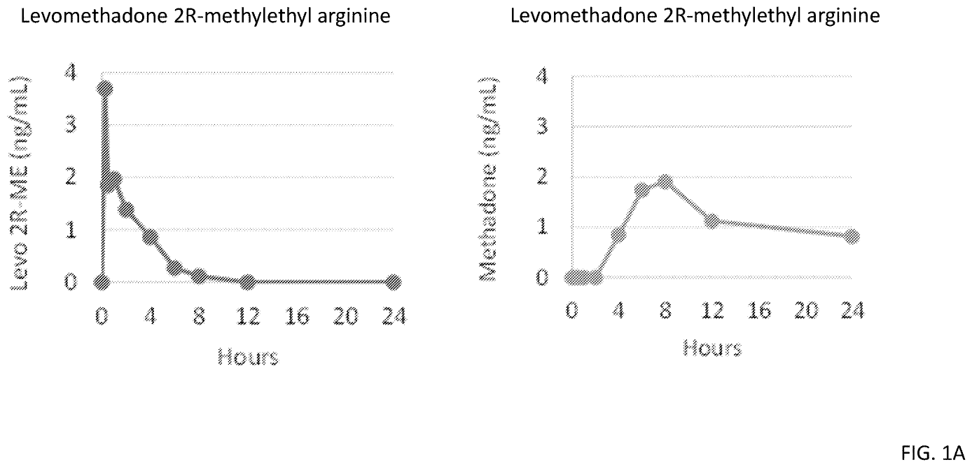

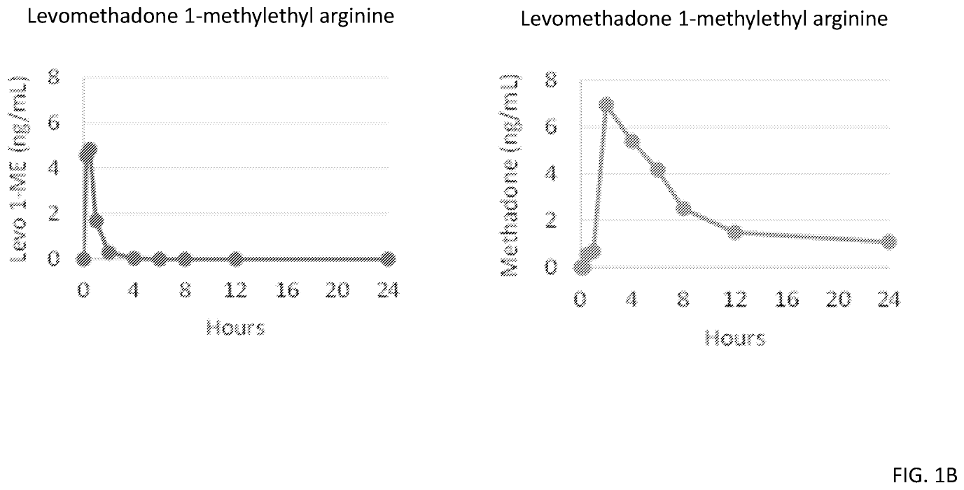

No clinical signs over the first 24 hours post dose were noted for any animal in any group. All four active agents were well tolerated at the 20 mg/kg dose level. Plasma sample concentration for the active agent and methadone was completed using a qualified LC-MS/MS method for each compound. FIGS. 1A-1B depict the concentration versus time of methadone active agents and methadone after the single 20 mg/kg oral dose. FIG. 1A depicts the concentration of Levomethadone 2R-methylethyl arginine and methadone over a 24 hour period after administration. FIG. 1B depicts the concentration of Levomethadone 1-methylethyl arginine and methadone over a 24 hour period after administration.

### Intravenous Administration

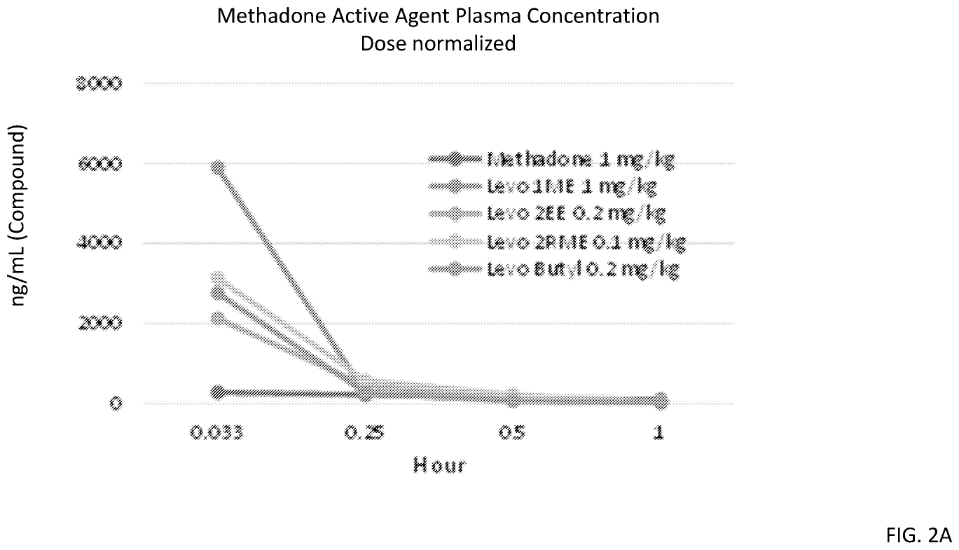

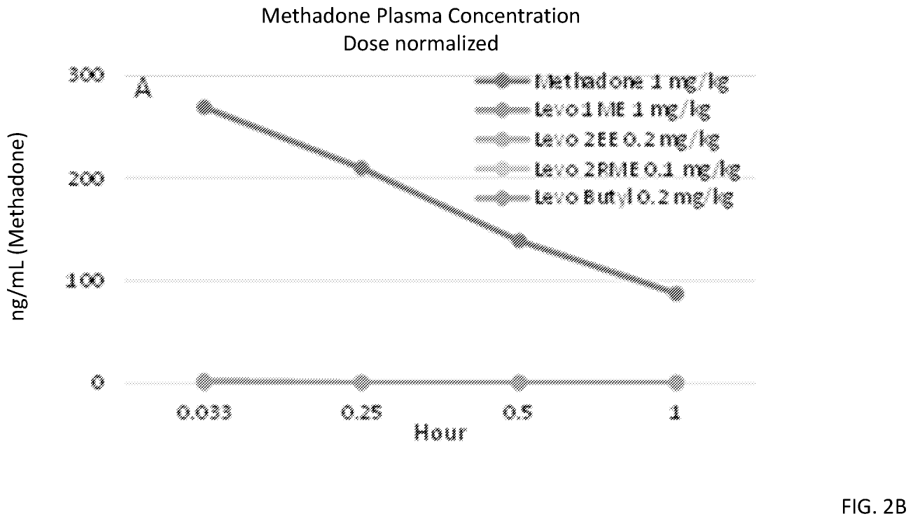

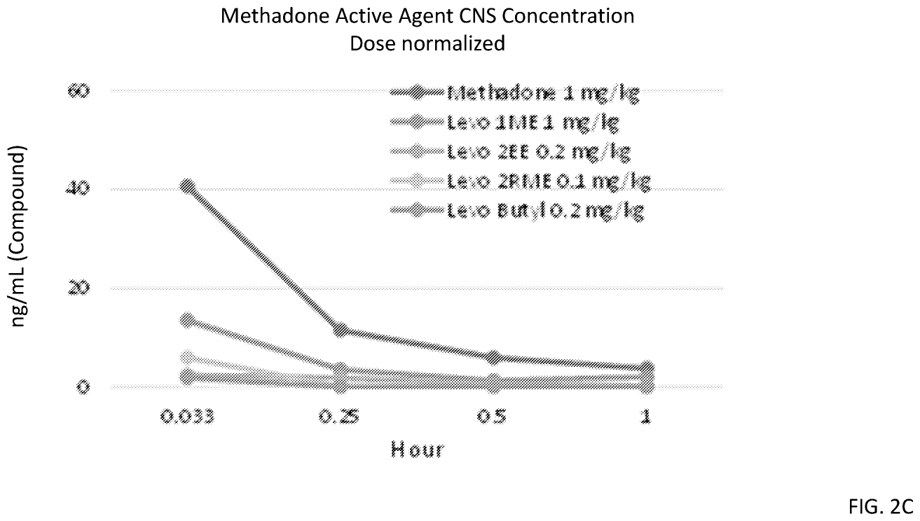

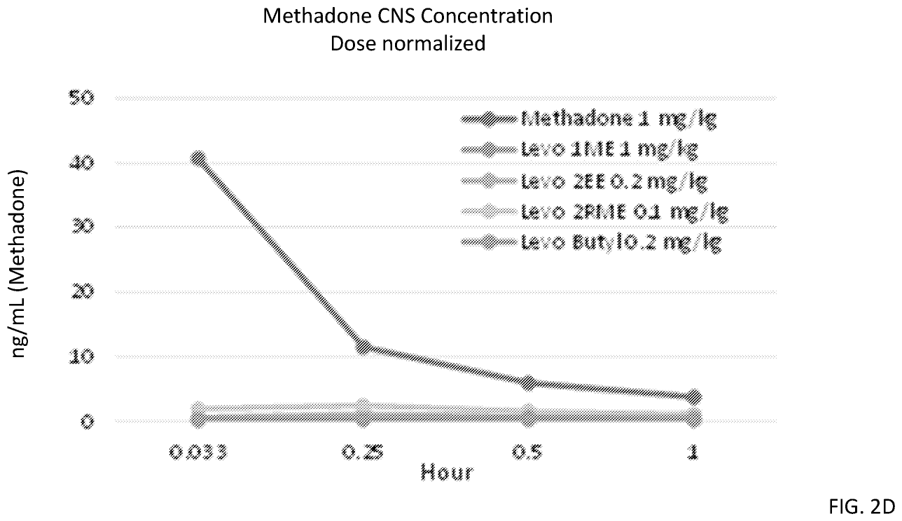

A Pharmacokinetic Study of Methadone and Two Analogues (IV) in Male Sprague Dawley Rats The objective of this study was to collect samples for the determination of the pharmacokinetics of Methadone and Methadone active agents following intravenous (IV) administration in rats. Methadone and four different methadone active agents were evaluated for conversion to methadone. FIGS. 2A-2D depict dose normalized plasma and cerebrospinal fluid (CSF) concentrations of methadone and methadone active agent after a single intravenous bolus administration according to certain embodiments. As shown in FIGS. 2A-2D, the methadone active agents did not convert to methadone following IV administration (FIGS. 2B and 2D). The methadone active agents also did not penetrate the CNS to the same extent as methadone (dose normalized) (FIGS. 2A and 2C).

### Safety Studies

### Maximally Tolerated Dose Study of Methadone and Methadone Active Agents by Oral Gavage Administration in Sprague Dawley Rats

The tolerability of each methadone active agent (Levo 1-ME, Levo 2R-ME, Levo 2-EE, and Levo-Butyl) was assessed after a single dose at 50, 75, 100, and 125 mg/kg. Methadone was also tested at the approximate molar equivalent doses (25, 37.5, 50 and 62.5 mg/kg) in a single male rat at each dose level. A minimum of one hour observation was completed before moving to the next highest dose level. Observations were completed on each rat at 15 min, and 1, 2, 3, and 4 hours post dose. Each animal was euthanized 72 hours post dose. Due to limited supply of each active agent only one animal was tested at each dose level, and lack of a positive dose response that was observed for a number of the animals was anticipated to be due to variability between animals.

All five groups tolerated the highest dose tested and therefore the MTD dose was not reached in this study. No clinical signs were observed for the animal treated with methadone until the highest dose (62.5 mg/kg) where clinical observations of decreased activity, shallow breathing, fixed stare, low carriage were observed starting at the 1-hour time point and only decreased activity continued through the 4-hour observation timepoint.

Levo 2R-ME and the Levo-Butyl methadone active agents showed some observations at 50 and 75 mg/kg yet no clinical observations through 72 hr period or limited observations at 100 mg/kg and 125 mg/kg (decreased activity and closed eyes (at 3 hours). The inconsistency in clinical signs between doses is likely the result of the low animal numbers per dose.

Levo 1-ME and Levo 2-EE treated animals did not have any clinical observations up to doses of 100 mg/kg. Levo 2-EE orally doses animal displayed abnormal gait, decreased activity, and closed eyes at 125 mg/kg.

The data suggest there is significant variability between animals in how they respond to methadone and the methadone active agents. Higher doses and more animals per group will be required in future studies. This preliminary data suggests dose levels up to 125 mg/kg are well tolerated for all the methadone active agents with only transient clinical signs common to opioid administration being observed that resolved within 4 hours.

ScreenPatch® Assay to Study the Effects of Methadone and Methadone Active Agents on hERG Ion Channels Expressed in Mammalian Cells

The objective of this study was to examine the in vitro effects of methadone and the methadone active agents on the hERG (human ether-à-go-go-related gene) channel expressed in CHO cells. The test article effects were evaluated using SyncroPatch® 384PE systems (SP384PE; Nanion Technologies, Livingston, NJ). After establishment of a whole-cell configuration, membrane currents were recorded using patch clamp amplifier in the SP384PE system.

Test article (TA) concentrations were applied to naïve cells (n=4, where n=replicates/concentration). Each application consisted of addition of 40 μL of 2× concentrated test article solution to the total 80 μL of final volume of the extracellular well of the SP384PE chip. Duration of exposure to each compound concentration was five (5) minutes.

hERG current was measured using stimulus voltage patterns with fixed amplitudes: activation pre-pulse (TP1) to +40 mV for 2 s and test pulse (TP2) to −40 mV for 2 s from a holding potential of −80 mV.
hERG current was measured as the outward peak current at TP2 (tail current). The stimulation was repeated with 0.1 Hz frequency during 2 min as baseline and 5 min after TA application.

IC₅₀ values of block for each compound are presented Table 1. In cases where maximal blocking effect was less than 50%, the IC₅₀ value was not calculated. In conclusion, all of the methadone active agents demonstrated decreased hERG ion channel blocking and therefore would be expected to have less cardiotoxicity.

TABLE 1
IC₅₀ values of hERG ion channel block
with test articles and positive control

| | | | Methadone |
|---|---|---|---|
| | | IC₅₀, | Active Agent/ |
| Group | Compound | μM | Methadone Ratio |
| 1 | Levo 2RME active agent | 29.442 | 15.6 |
| 2 | Levo-Butyl active agent | 51.465 | 27.3 |
| 3 | Levo 2EE active agent | >100 | >53 |
| 4 | Levo 1ME active agent | >100 | >53 |
| 5 | Racemic 2ME active agent | 36.885 | 19.6 |
| 6 | Racemic Butyl active agent | 13.200 | 7.0 |
| 7 | Racemic 2EE active agent | >100 | >53 |
| 8 | Racemic 1ME active agent | >100 | >53 |
| 9 | Racemic Methadone HCl | 1.885 | — |
| PC | Cisapride (PC) | 0.018 | NA |

Racemic methadone was the most potent hERG channel blocker with an IC₅₀ 1.885 μM. In comparison, decreased hERG activity was observed for all the levo-methadone active agents as well as the racemic methadone active agents tested (Table 3). levo-methadone active agents designated 1-ME and 2-EE were found to have the least effect with IC₅₀'s greater than 100 μM as did their racemic counterpart. This is an important finding suggesting decreased cardio toxicity by these levo-methadone active agents.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

What is claimed is:

1. A compound of formula ME-(I): 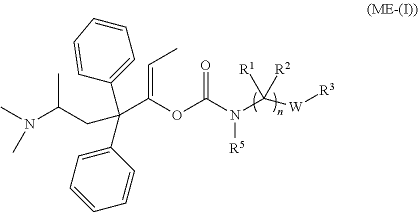
wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 2 to 4;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

2. The compound of claim 1, wherein R⁵ is hydrogen.

3. The compound of claim 1, wherein R⁵ is methyl or ethyl.

4. The compound of any one of claim 1-3, wherein R¹ and R² are hydrogen.

5. The compound of any one of claims 1-4, wherein R³ is hydrogen.

6. The compound of any one of claims 1-5, wherein R³ is methyl or ethyl.

7. A compound of formula ME-(II): 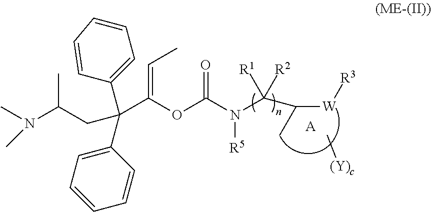
wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

8. A compound of formula ME-(III): 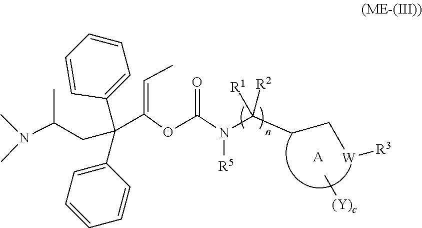
wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

9. A compound of formula ME-(IV): 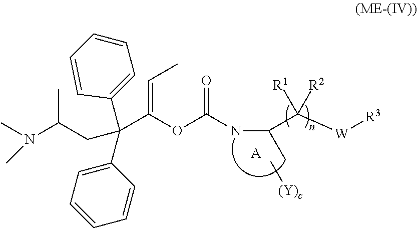
wherein:
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 1 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

10. A compound of formula ME-(IV): 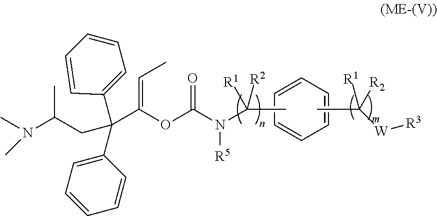
wherein:
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl;
or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 0 to 4;
m is an integer from 0 to 4;
the A ring is a heterocyclic 5 to 12-membered ring;
each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;
c is a number from zero to 3;
R³ is hydrogen, alkyl, substituted alkyl, acyl, substituted acyl, alkoxycarbonyl, substituted alkoxycarbonyl, arylalkyl, substituted arylalkyl, aryl and substituted aryl;
W is independently a bond, —CH₂—, —NR⁸—, —O— or —S—;
R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
or a salt, hydrate or solvate thereof.

11. A compound selected from:
a) (R, E)-6-(dimethylamino)-4,4-diphenylhept-2-en-3-yl (1-aminopropan-2-yl)(methyl)carbamate: 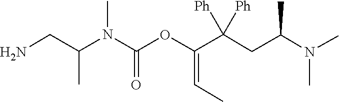 [SMILES: C/C=C(/OC(=O)N(C)C(C)CN)C(C[C@@H](C)N(C)C)(c1ccccc1)c1ccccc1]
or a salt, hydrate or solvate thereof;
b) (R, E)-6-(dimethylamino)-4,4-diphenylhept-2-en-3-yl ((R)-2-aminopropyl)(methyl)carbamate: 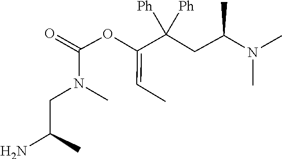 [SMILES: C/C=C(/OC(=O)N(C)C[C@@H](C)N)C(C[C@@H](C)N(C)C)(c1ccccc1)c1ccccc1]
or a salt, hydrate or solvate thereof;
c) (R, E)-6-(dimethylamino)-4,4-diphenylhept-2-en-3-yl ((S)-2-aminopropyl)(methyl)carbamate: 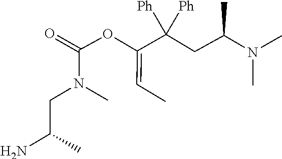 [SMILES: C/C=C(/OC(=O)N(C)C[C@H](C)N)C(C[C@@H](C)N(C)C)(c1ccccc1)c1ccccc1]
or a salt, hydrate or solvate thereof.
d) (R, E)-6-(dimethylamino)-4,4-diphenylhept-2-en-3-yl (4-aminobutyl)(methyl)carbamate: 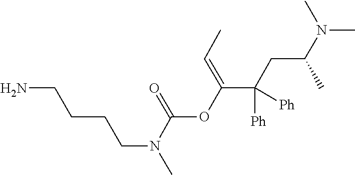 [SMILES: C/C=C(/OC(=O)N(C)CCCCN)C(C[C@@H](C)N(C)C)(c1ccccc1)c1ccccc1]
or a salt, hydrate or solvate thereof; or
e) (R,E)-6-(dimethylamino)-4,4-diphenylhept-2-en-3-yl(2-aminobutyl)(methyl)carbamate: 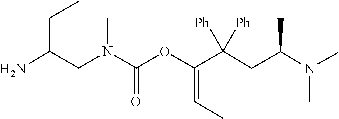 [SMILES: C/C=C(/OC(=O)N(C)CC(N)CC)C(C[C@@H](C)N(C)C)(c1ccccc1)c1ccccc1]
or a salt, hydrate or solvate thereof.

12. A composition comprising:
a compound according to any one of claims 1-11; and
a pharmaceutically acceptable carrier.

13. A method comprising administering to a subject in need thereof a compound of any one of claims 1-11 or a composition of claim 12.
